(19) United States
(12) Patent Application Publication
(10) Pub. No.: US 2025/0241983 A1
(43) Pub. Date: Jul. 31, 2025

(54) NON-OPIOID METHODS FOR TREATING PAIN

(71) Applicant: UNIVERSITY OF CONNECTICUT, Farmington, CT (US)

(72) Inventors: Tannin SCHMIDT, Farmington, CT (US); Sumit YADAV, Farmington, CT (US); Bin FENG, Farmington, CT (US); Sharareh EMADI, Farmington, CT (US)

(21) Appl. No.: 18/685,958

(22) PCT Filed: Aug. 29, 2022
(86) PCT No.: PCT/US2022/041820

§ 371 Date: Feb. 23, 2024

Related U.S. Application Data
(60) Provisional application No. 63/238,724, filed on Aug. 30, 2021.

(51) Int. Cl. A61K 38/17; A61P 25/04

(52) U.S. Cl. CPC A61K 38/1709; A61P 25/04

(57) ABSTRACT

The present disclosure relates to administration of pharmaceutically acceptable composition as a means to reduce acute and chronic pain and treat other conditions. Pharmaceutical acceptable composition comprising a pharmaceutically effective amount of a full length recombinant human PRG4 (rhPRG4) or a fragment thereof and a pharmaceutically acceptable carrier or diluent.

### CROSS-REFERENCE TO RELATED APPLICATIONS

This application claims the benefit of U.S. Provisional Patent Application No. 63/238,724, filed on Aug. 30, 2021, the contents of which is incorporated herein by reference in its entirety.

### STATEMENT OF GOVERNMENT SUPPORT

This invention was made with government support under grant numbers K08DE025914 and U01NS113873 awarded by the National Institutes of Health (NIH). The government has certain rights in the invention.

### TECHNICAL FIELD

In some aspects, the present disclosure is directed to methods of treating pain or inhibiting tumor necrosis factor (TNF)-α simulated nerve growth factor (NGF), transient receptor potential ankyrin 1 (TrpA1) expression, and/or nerve growth factor (NGF)/tropomyosin receptor kinase A (TrkA) binding by administering PRG4 to a subject in need thereof.

### BACKGROUND

Pain of various types is a leading cause of discomfort and disability in the United States today. Pain associated with the musculoskeletal system can arise from multiple causes including over-exertion, trauma, and disease. For example, pain from diseases such as arthritis are increasingly problematic as the average age of the population rises. Left untreated, arthritic pain can severely compromise an affected individual's quality of life.

Currently available pharmacological therapies target palliation of pain and include analgesics (e.g., acetaminophen, cyclooxygenase-2-specific inhibitors, non-selective nonsteroidal anti-inflammatory drugs (NSAIDs, such as ibuprofen, naproxen sodium, aspirin), opioids (e.g., codeine, morphine, hydrocodone, hydromorphone, oxycodone), and synthetic opioids (e.g., fentanyl, tramadol, nalbuphine, pethidine). However, the clinical presentation of osteoarthritis is usually monoarticular or oligoarticular with fluctuations in intensity and localization over time. It is therefore logical to consider local therapeutic modalities in order to avoid untoward systemic effects. Several compounds have been used intra-articularly (e.g., glucocorticoids, hyaluronic acid) or topically (e.g., capsaicin, methylsalicylate). However, for example, the benefit of intra-articular glucocorticoids lasts only a few days (Barron, M. C., 2007, J. Am. Osteopath., 107, ES21-27).

However, many of the currently available pharmacological therapies for the treatment of pain have undesirable side effects. For example, long-term use of NSAIDs can lead to undesirable consequences including gastric ulcers, kidney damage, hearing loss, and inhibition of cartilage formation. In addition, many people are sensitive to NSAIDs and cannot take them for pain relief due to ancillary medical conditions. While other classes of pain relievers (e.g., opioids and synthetic opioids) carry a risk of abuse or tolerance. Accordingly, there remains a need in the art for new and effective treatments to safely and effectively reduce acute and/or chronic pain.

### SUMMARY

The present disclosure is directed to administration a pharmaceutically effective amount of a full length recombinant human PRG4 (rhPRG4) or a fragment thereof as a means to reduce acute and chronic pain to a subject in need of treatment thereof (e.g., a subject suffering from pain, such as acute or chronic pain).

In one aspect, disclosed herein is a method of treating pain, wherein the method comprises administering a pharmaceutically effective amount of PRG4 or a fragment thereof to a subject in need thereof. In some embodiments, PRG4 is rhPRG4. In some embodiments, PRG4 is administered to the subject as a pharmaceutically acceptable composition. In some embodiments, the pharmaceutically acceptable composition comprises a pharmaceutically acceptable carrier or diluent.

In certain embodiments, the pain is joint pain. For example, the joint pain can be associated with arthritis, including osteoarthritis, rheumatoid arthritis (RA), juvenile idiopathic arthritis, fibromyalgia, gonococcal arthritis, gout, lupus, psoriatic arthritis, reactive arthritis, septic arthritis, or a combination thereof. Alternatively, the joint pain can be associated with a temporomandibular joint (TMJ) disorder or dry mouth. In some such embodiments, the temporomandibular joint (TMJ) disorder is TMJ arthritis, erosion of the joint, habitual grinding or clenching of the teeth, structural problems at birth, the use of orthodontic braces, poor posture that strains the muscles of the neck and face, prolonged stress, poor diet, lack of sleep or a combination thereof.

In other embodiments, the joint pain is associated with a strain, a sprain, a broken bone, or a dislocation.

In yet other embodiments, the joint pain is associated with bursitis.

In other embodiments, the joint pain is associated with a meniscal tear, anterior cruciate ligament (ACL) tear, or a rotator cuff tear.

In still other embodiments, the joint pain is associated with Still's disease, ankylosing spondylitis, avascular necrosis, bone cancer, complex regional pain syndrome, hypothyroidism, leukemia, Lyme disease, osteomyelitis, Paget's disease of bone, polymyalgia rheumatica, pseudogout, rheumatic fever, rickets, sarcoidosis, tendinitis, or a combination thereof.

In certain embodiments, the pain is inflammatory bowel disease pain. In some embodiments, the subject has Crohn's disease. In other embodiments, the subject has ulcerative colitis.

In certain embodiments, the subject has interstitial cystitis/bladder pain syndrome (IC/BPS).

In certain embodiments, the pain is neuropathic pain. In some embodiments, the subject has neuropathic pain caused by nerve pressure or nerve damage after surgery or trauma, a viral infection, cancer, a vascular malformation, alcoholism, a neurological condition such as multiple sclerosis, a metabolic condition, or a combination thereof. In other embodiments, the subject has neuropathic pain caused by metabolic condition, wherein the metabolic condition is diabetes. In yet other embodiments, the neuropathic pain is a side effect of a medication.

In certain embodiments, the pain is inflammatory pain. In some embodiments, the inflammatory pain is acute inflammatory pain. In some embodiments, the inflammatory pain is constant.

In certain embodiments, the pain is orofacial pain. In some embodiments, the orofacial pain is caused by a temporomandibular disorder (TMJD), a trigeminal neuropathic pain disorder, a neurovascular disorder, a complex temporomandibular disorder, burning mouth syndrome, a sleep disorder, orofacial dyskinesia and/or dystonia, trauma, cervicalgia, or a combination thereof.

In certain embodiments, the pain is migraine pain.

In certain embodiments, the pain is cancer pain. In one embodiment, the cancer pain is caused by the cancer itself. In another embodiment, the cancer pain occurs or results if the cancer grows into or destroys nearby tissue. In yet another embodiment, the cancer pain is the result of the growth of the tumor which t can press on nerves, bones, or organs. In still yet another embodiment, the pain is caused by the tumor which can also release chemicals. In yet a further embodiment, the cancer pain includes, but is not limited to, a pain that is dull, achy, sharp, burning, or any combination thereof. In still yet a further embodiment, the cancer pain can be constant, intermittent, mild, moderate, severe, or any combination thereof.

In certain embodiments, the pain is ocular neuropathic pain. In some embodiments, the ocular neuropathic pain is associated with damaged or dysfunctional corneal nerves. In some embodiments, the ocular neuropathic pain is caused by peripheral or centralized sensitization. In some embodiments, the subject having ocular neuropathic pain also has dry eye.

In certain embodiments, the pain is Morton's neuroma pain. In some embodiments, the Moton's neuroma pain is associated with a thickening of the tissue that surrounds the digital nerve leading to the toes.

In certain embodiments, the pain is a general pain disease. In some embodiments, the general pain disease is a chronic pain syndrome. For example, the chronic pain syndrome can be associated with osteoarthritis, rheumatoid arthritis (RA), back pain, fibromyalgia, inflammatory bowel disease (IBD), surgical trauma, advanced cancer, or a combination thereof.

In some embodiments the chronic pain syndrome is chronic pelvic pain.

In some embodiments, the chronic pain syndrome is associated with endometriosis.

In another aspect, disclosed herein is a method of inhibiting tumor necrosis factor (TNF)-α simulated nerve growth factor (NGF) by administering a pharmaceutically effective amount of PRG4 or a fragment thereof to a subject in need of treatment thereof. In some embodiments, PRG4 is rhPRG4. In some embodiments, the PRG4 is administered to the subject as a pharmaceutically acceptable composition. In some embodiments, the pharmaceutically acceptable composition comprises a pharmaceutically acceptable carrier or diluent.

In yet another aspect, disclosed herein is a method of inhibiting transient receptor potential ankyrin 1 (TrpA1) expression by administering a pharmaceutically effective amount of PRG4 or a fragment thereof to a subject in need of treatment thereof. In some embodiments, PRG4 is rhPRG4. In some embodiments, PRG4 is administered to the subject as a pharmaceutically acceptable composition. In some embodiments, the pharmaceutically acceptable composition comprises a pharmaceutically acceptable carrier or diluent.

In still another aspect, disclosed herein is a method of inhibiting nerve growth factor (NGF)/tropomyosin receptor kinase A (TrkA) binding by administering a pharmaceutically effective amount of PRG4 or a fragment thereof to a subject in need of treatment thereof. In some embodiments, PRG4 is rhPRG4. In some embodiments, PRG4 is administered to the subject as a pharmaceutically acceptable composition. In some embodiments, the pharmaceutically acceptable composition comprises a pharmaceutically acceptable carrier or diluent.

### BRIEF DESCRIPTION OF THE DRAWINGS

FIG. 1A provides a graph showing the TrkA-rhPRG4 binding kinetics.

FIG. 1B provides a graph showing the TrkA-rhPRG4 binding affinity.

FIG. 2A shows expression and localization of NGF in PRG4 deficient mice (GT).

FIG. 2B shows expression and localization of NGF in TMX treated mice (GTT).

FIG. 3A shows expression of TrkA in PRG4 deficient mice (GT).

FIG. 3B shows expression of TrkA in TMX treated mice (GTT).

FIG. 4A shows a view of dorsal root ganglia (DRG) cell 1 for assessing NGF sensitization of CAP response as shown in FIG. 4B.

FIG. 4B shows NGF sensitization of capsaicin (CAP) response. The peak response is increased as the effect of NGF application.

FIG. 5A shows a view of DRG cell 1 for assessing NGF sensitization of CAP response as shown in FIG. 5B.

FIG. 5B shows NGF sensitization of capsaicin (CAP) response. The decay time constant is increased as an effect of NGF application.

FIG. 6 shows a view of DRG cells 1 to 3 for assessing NGF sensitization of CAP response by pre-application of lubricin as shown in FIGS. 7, 8 and 9.

FIG. 7 shows NGF sensitization of CAP response by pre-application of lubricin. The NGF sensitization of CAP response is blunted by pre-application of lubricin.

FIG. 8 shows NGF sensitization of CAP response by pre-application of lubricin. The NGF sensitization of CAP response is blunted by pre-application of lubricin.

FIG. 9 shows NGF sensitization of CAP response by pre-application of lubricin. The NGF sensitization of CAP response is blunted by pre-application of lubricin.

FIG. 10A shows a view of DRG cell 1 for assessing NGF sensitization of CAP response by post-application of lubricin as shown in FIG. 10B.

FIG. 10B shows NGF sensitization of CAP response by post-application of lubricin. The NGF sensitization of CAP response is slighted reduced by post-application of lubricin.

FIG. 11A shows a view of DRG cell 1 for assessing NGF sensitization of CAP response by co-application of NGF and lubricin as shown in FIG. 11B.

FIG. 11B shows NGF sensitization of CAP response by co-application of NGF and lubricin. The NGF sensitization of CAP response is prevented when NGF and lubricin are added together.

FIG. 12 shows NGF sensitization of CAP response. The decay time constant is increased as the effect of NGF application.

FIG. 13 shows NGF sensitization of CAP response. The decay time constant is increased as the effect of NGF application.

FIG. 14 shows NGF sensitization of CAP response by pre-application of lubricin. The NGF sensitization of CAP response is blunted by pre-application of lubricin.

FIG. 15 shows NGF sensitization of CAP response by pre-application of lubricin. The NGF sensitization of CAP response is blunted by pre-application of lubricin.

FIG. 16 shows NGF sensitization of CAP response by pre-application of lubricin. The NGF sensitization of CAP response is blunted by pre-application of lubricin.

FIG. 17 shows NGF sensitization of CAP response by pre-application of lubricin. The NGF sensitization of CAP response is blunted by pre-application of lubricin.

FIG. 18 shows NGF sensitization of CAP response by pre-application of lubricin. The NGF sensitization of CAP response is blunted by pre-application of lubricin. Thus, FIGS. 7-9 and 14-18 show that the pre-treatment with lubricin inhibits/prevents NGF sensitization of capsaicin-evoked intracellular calcium response in DRG.

FIG. 19A shows NGF sensitization of capsaicin (CAP) response. The peak response is increased as the effect of NGF application.

FIG. 19B shows NGF sensitization of capsaicin (CAP) response. The decay time constant is increased as the effect of NGF application.

FIG. 20 shows that PRG4 prevents the sensitization of DRG neurons by NGF. FIG. 20A shows ratiometric calcium imaging conducted on dissociated DRG neurons loaded with the fluorescent dye Fura2. The rise in intracellular calcium [Ca²⁺]ᵢ was caused by a brief capsaicin perfusion (CAP, 1 μM for 8 sec), which is quantified by the ratio of evoked fluorescent intensity of 340 and 380 nm excitation, respectively (F340/F380). FIG. 20B shows that application of NGF (0.1 μg/mL for 7 min) increases the peak and duration of CAP-evoked [Ca²⁺]ᵢ transients. FIG. 20C shows that pre-treatment of PRG4 (0.3 mg/mL for 7 min) prevents the NGF sensitization of CAP responses.

FIG. 21A shows pain measurement data via Von Frey Test on male wild type mice (WT) and PRG4 deficient mice (GT). FIG. 21A shows that PRG4 deficient mice (GT) are much more sensitive to pain.

FIG. 21B shows pain measurement data via Von Frey Test on female wild type mice (WT) and PRG4 deficient mice (GT). FIG. 21B shows that PRG4 deficient mice (GT) are much more sensitive to pain.

### DETAILED DESCRIPTION

Approximately 20 to 30% of adults suffer from refractory pain such as chronic low back pain that cannot be suitably alleviated even with the use of various analgesics. This is a large societal issue as it leads to a decreased productivity (Elzahaf et al., Curr Med Res Opin. 2012; 30:1221-29). The development of novel analgesics is, therefore, desirable. Nerve growth factors (NGFs) on the peripheral nociceptive neuron terminals bind to tropomyosin receptor kinase (Trk) A (“TrkA”) on the cell membrane, which are then taken up by endosomes and are subsequently transported in a retrograde manner through the axon to the dorsal root ganglia cell bodies. There, the downstream intracellular signal transduction system is activated, producing various types of proteins (Delcroix et al., Neuron, 2003; 39:69-84). NGFs secreted by inflammatory cells act on TrkA located on the cell membrane of the sensory nerve endings, phosphorylating other proteins associated with pain, which induces a conformational change and increases the expression of these proteins. It is believed that these in turn elicit peripheral sensitization in the peripheral nerve and central sensitization in the spinal cord, inducing the onset of a hypersensitive reaction (hyperalgesia) in response to pain, together with allodynia a painful response to a stimulus that is not normally painful (Woolf et al., Pro Natl Acad Sci USA, 1999; 96:7723-30; Zhuang et al., J Neurosci., 2004; 24:8300-09; Ueda H., Mol Pain, 2008; 4:11). Accordingly, NGF/TrkA signaling is involved in both nociceptive and neuropathic pain, and NGF/TrkA is one of the important targets for such therapeutic development (Hefti et al., Trends Pharmacol Sci., 2006; 27:85-91.30-32; Watson et al., BioDrugs, 2008; 22:349-59; Xian et al., Nat Clin Pract Rheumatol., 2009; 5:92-98).

Disclosed herein are methods for the treatment of pain. Specifically, disclosed herein is rhPRG4 as a treatment for pain. It is shown herein that rhPRG4 binds high affinity NGF receptor TrkA. Additionally shown herein is that rhPRG4 inhibits tumor necrosis factor (TNF)-α simulated NGF and transient receptor potential ankyrin 1 (TrpA1) expression in human corneal epithelial cells.

Also disclosed herein is that PRG4 deficient mice show increased expression of NGF and TrkA. Moreover, PRG4 inhibits NGF sensitization of capsaicin response in dorsal root ganglia (DRG).

Also disclosed herein is that PRG4 deficient mice have increased pain sensitivity compared to wild type mice.

Also disclosed herein is that PRG4 inhibits the sensitization of TrpV1 channels by NGF.

Also disclosed herein is that PRG4 competitively binds to TrkA to inhibit the NGF-induced sensitization of TrpV1 channels.

Also disclosed herein is that PRG4 inhibits the expression of NGF and TrkA via inhibition of NF-Kappa B.

### 1. Definitions

Throughout the present specification and the accompanying claims, the words “comprise,” “include,” and “have” and variations thereof such as “comprises,” “comprising,” “includes,” “including,” “has,” and “having” are to be interpreted inclusively. That is, these words are intended to convey the possible inclusion of other elements or integers not specifically recited, where the context allows. No language in the specification should be construed as indicating any non-claimed element essential to the practice of the disclosure.

The terms “a,” “an,” and “the” and similar referents used in the context of describing the disclosure (especially in the context of the following claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, each individual value is incorporated into the specification as if it were individually recited herein. Ranges may be expressed herein as from “about” (or “approximately”) one particular value, and/or to “about” (or “approximately”) another particular value. When such a range is expressed, another embodiment includes from the one particular value and/or to the other particular value. Similarly, when values are expressed as approximations, by use of the antecedent “about” or “approximately” it will be understood that the particular value forms another embodiment. It will be further understood that the endpoints of each of the ranges are disclosed both in relation to the other endpoint, and independently of the other endpoint.

All methods described herein can be performed in any suitable order unless otherwise indicated herein or otherwise clearly contradicted by context. Further, all methods described herein and having more than one step can be performed by more than one person or entity. Thus, a person or an entity can perform step (a) of a method, another person or another entity can perform step (b) of the method, and a yet another person or a yet another entity can perform step (c) of the method, etc. The use of any and all examples, or exemplary language (e.g., “such as”) provided herein is intended merely to better illuminate the disclosure and does not pose a limitation on the scope of the disclosure otherwise claimed.

Units, prefixes, and symbols are denoted in their Systeme International de Unites (SI) accepted form. Unless otherwise indicated, nucleic acids are written left to right in 5′ to 3′ orientation; amino acid sequences are written left to right in amino to carboxy orientation.

Groupings of alternative elements or embodiments of the disclosure disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is herein deemed to contain the group as modified thus fulfilling the written description of all Markush groups used in the appended claims.

The headings used herein are for organizational purposes only and are not meant to be used to limit the scope of the description or the claims, which can be had by reference to the specification as a whole. Accordingly, the terms defined immediately below are more fully defined by reference to the specification in its entirety.

Illustrations are for the purpose of describing a preferred embodiment of the disclosure and are not intended to limit the disclosure thereto.

As used herein, the term “about” refers to a range of values of plus or minus 10% of a specified value. For example, the phrase “about 200” includes plus or minus 10% of 200, or from 180 to 220, unless clearly contradicted by context.

As used herein, the term “administering” means the actual physical introduction of a composition into or onto (as appropriate) a host or cell. Any and all methods of introducing the composition into the host or cell are contemplated according to the disclosure; the method is not dependent on any particular means of introduction and is not to be so construed. Means of introduction are well-known to those skilled in the art, and also are exemplified herein.

The terms “modulate,” “modulation,” or “modulating” are art-recognized and refer to up-regulation (i.e., activation, stimulation, increase), or down-regulation (i.e., inhibition, suppression, reduction, or decrease) of a response, or the two in combination or apart.

As used herein, “optional” or “optionally” means that the subsequently described event or circumstance may or may not occur, and that the description includes instances where said event or circumstance occurs and instances where it does not.

As used herein, the term “pharmaceutically acceptable” refers to compositions that are physiologically tolerable and do not typically produce an allergic or similar untoward reaction when administered to a subject, preferably a human subject. Preferably, as used herein, the term “pharmaceutically acceptable” means approved by a regulatory agency of a federal or state government or listed in the U.S. Pharmacopeia or other generally recognized pharmacopeia for use in animals, and more particularly in humans.

As used herein, the term “substantially decreased” and grammatical equivalents thereof refer to a level, amount, concentration of a parameter, such as a chemical compound, a metabolite, a nucleic acid, a polypeptide or a physical parameter (pH, temperature, viscosity, etc.) measured in a sample that has a decrease of at least 10%, preferably about 20%, more preferable about 40%, even more preferable about 50% and still more preferably a decrease of more than 75% when compared to the level, amount, or concentration of the same chemical compound, nucleic acid, polypeptide or physical parameter in a control sample.

As used herein, the term “substantially increased” and grammatical equivalents thereof refer to a level, amount, concentration of a parameter, such as a chemical compound, a metabolite, a nucleic acid, a polypeptide or a physical parameter (pH, temperature, viscosity, etc.) measured in a sample that has an increase of at least 30%, preferably about 50%, more preferable about 75%, and still more preferably an increase of more than 100% when compared to the level, amount, or concentration of the same chemical compound, nucleic acid, polypeptide, or physical parameter in a control sample.

As used herein, the term “subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (for example, a monkey, such as a cynomolgous or rhesus monkey, chimpanzee, etc.) and a human). In some embodiments, the subject may be a human or a non-human. In some embodiments, the subject is a human. The subject or patient may be undergoing other forms of treatment. In some embodiments, the subject is a human that may be undergoing other forms of treatment.

As used herein, the terms “treat,” “treating,” and “treatment” include inhibiting the pathological condition, disorder, or disease, e.g., arresting or reducing the development of the pathological condition, disorder, or disease or its clinical symptoms; or relieving the pathological condition, disorder, or disease, e.g., causing regression of the pathological condition, disorder, or disease or its clinical symptoms. These terms also encompass therapy and cure. Treatment means any way the symptoms of a pathological condition, disorder, or disease are ameliorated or otherwise beneficially altered. Preferably, the subject in need of such treatment is a mammal, preferably a human.

### 2. PRG4 Protein

PRG4, also referred to as lubricin, is a lubricating polypeptide, which in humans is expressed from the megakaryocyte stimulating factor (MSF) gene, also known as PRG4 (see NCBI Accession Number AK131434-U70136). Lubricin is a ubiquitous, endogenous glycoprotein that coats the articulating surfaces of the body. Lubricin is highly surface active molecule (e.g., holds onto water), that acts primarily as a potent cytoprotective, anti-adhesive and boundary lubricant. The molecule has a long, central mucin-like domain located between terminal protein domains that allow the molecule to adhere and protect tissue surfaces. Its natural form, in all mammals investigated, contains multiple repeats of an amino acid sequence which is at least 50% identical to KEPAPTT. Natural lubricin typically comprises multiple redundant forms of this repeat, which typically includes proline and threonine residues, with at least one threonine being glycosylated in most repeats. The threonine anchored O-linked sugar side chains are critical for lubricin's boundary lubricating function. The side chain moiety typically is a β(1-3)Gal-GalNAc moiety, with the β(1-3)Gal-GalNAc typically capped with sialic acid or N-acetylneuraminic acid. The polypeptide also contains N-linked oligosaccharides. The gene encoding naturally occurring full length lubricin contains 12 exons, and the naturally-occurring MSF gene product contains 1,404 amino acids (including the secretion sequence) with multiple polypeptide sequence homologies to vitronectin including hemopexin-like and somatomedin-like regions. Centrally located exon 6 contains 940 residues. Exon 6 encodes the repeat rich, O-glycosylated mucin-like domain.

The amino acid sequence of the protein backbone of lubricin may differ depending on alternative splicing of exons of the human MSF gene. This robustness against heterogeneity was exemplified when researchers created a recombinant form of lubricin missing 474 amino acids from the central mucin domain, yet still achieved reasonable, although muted, lubrication (Flannery et al., Arthritis Rheum., 2009; 60(3):840-7). PRG4 has been shown to exist not only as a monomer but also as a dimer and multimer disulfide-bonded through the conserved cysteine-rich domains at both N- and C-termini. Lubris, LLC has developed a full-length recombinant form of human lubricin. The molecule is expressed using the Selexis Chinese hamster ovary cell line (CHO-M), with a final apparent molecular weight of about 450 to about 600 kDa, with polydisperse multimers frequently measuring at about 1,000 kDa or more, all as estimated by comparison to molecular weight standards on SDS tris-acetate 3-8% polyacrylamide gels. Of the total glycosylations, about half comprise two sugar units (GalNAc-Gal), and half three sugar units (GalNAc-Gal-Sialic acid). This method of recombinant human PRG4 production is disclosed in International Patent Publication No. WO 2015/061488.

Any one or more of various native and recombinant PRG4 proteins and isoforms may be utilized in the various embodiments described herein. For instance, U.S. Pat. Nos. 6,433,142; 6,743,774; 6,960,562; 7,030,223, and 7,361,738 disclose how to make various forms of human PRG4 expression product. In some aspects, a full length, glycosylated, recombinant PRG4, or lubricin, expressed from CHO cells can be used. This protein comprises 1,404 amino acids including a central exon comprising repeats of the sequence KEPAPTT variously glycosylated with O-linked β (1-3) Gal-GalNAc oligosaccharides, and including N- and C-terminal sequences with homology to vitronectin. The molecule is polydisperse with the glycosylation pattern of individual molecules varying, and can comprise monomeric, dimeric, and multimeric species.

As used herein, the term “PRG4” is used interchangeably with the term “lubricin.” Broadly, these terms refer to any functional isolated or purified native or recombinant PRG4 proteins, homologs, functional fragments, isoforms, and/or mutants thereof. All useful molecules comprise the sequence encoded by exon 6, or homologs or truncated versions thereof, for example, versions with fewer repeats within this central mucin-like KEPAPTT-repeat domain, preferably together with O-linked glycosylation. All useful molecules also comprise at least the biological active portions of the sequences encoded by exons 1-5 and 7-12, i.e., sequences responsible for imparting to the molecule its affinity for ECM and endothelial surfaces. In certain embodiments, a PRG4 protein has an average molar mass of between 50 kDa and 500 kDa, in other aspects, between 224 to 467 kDa, comprising one or more biological active portions of the PRG4 protein, or functional fragments, such as a lubricating fragment, or a homolog thereof. In still other embodiments, a PRG4 protein comprises monomers of average molar mass of between about 220 kDa to about 280 kDa. In still further aspects of the embodiments described herein, the PRG4 that can be native or recombinant PRG4. In some aspects, the PRG4 is native human PRG4. In other aspects, the PRG4 is a recombinant human PRG4 (e.g., rhPRG4). In some aspects, the native or recombinant human PRG4 is full length native or recombinant human PRG4. In other aspects, the native or recombinant human PRG4 comprises a fragment of the full-length native or recombinant human PRG4. In some aspects, the PRG4 fragment has a length of 5 amino acids, 10 amino acids, 15 amino acids, 20 amino acids, 30 amino acids, 35 amino acids, 40 amino acids, 45 amino acids, 50 amino acids, 55 amino acids, 60 amino acids, 65 amino acids, 70 amino acids, 75 amino acids, 100 amino acids, 200 amino acids, 300 amino acids, 400 amino acids, 500 amino acids, 600 amino acids, 700 amino acids, 800 amino acids, 900 amino acids, 1000 amino acids, 1100 amino acids, 1200 amino acids, 1300 amino acids, or 1400 amino acids.

Methods for isolation, purification, and recombinant expression of proteins such as PRG4 protein (either native or recombinant) are well known in the art. In certain cases, the method starts with cloning and isolating mRNA and cDNA encoding PRG4 proteins or isoforms using standard molecular biology techniques, such as PCR or RT-PCR. The isolated cDNA encoding the PRG4 protein or isoform is then cloned into an expression vector and expressed in a host cell for producing recombinant PRG4 protein, and isolated from the cell culture supernatant. A method for production of recombinant human PRG4 is provided in International Publication No. WO 2015/061488.

### 3. Combination Therapies

According to one embodiment, PRG4 or a fragment thereof is administered to the patient in combination with a compound having analgesic properties. For example, PRG4 or a fragment thereof can be co-administered with a nonsteroidal anti-inflammatory drug, acetaminophen, an opioid, a steroid, a triptan, etc. The terms “co-administer,” “co-administered,” and “co-administration,” as used herein, mean the administration of two or more agents within 24 hours of each other, e.g., as part of a treatment regimen. In other embodiments, “co-administer,” “co-administered,” and “co-administration,” refer to administration within 2 hours of each other. In other embodiments, “co-administer,” “co-administered,” and “co-administration,” refers to administration within 30 minutes of each other. In other embodiments, “co-administer,” “co-administered,” and “co-administration,” refers to administration within 15 minutes of each other. In other embodiments, “co-administer,” “co-administered,” and “co-administration,” refers to simultaneous administration as either part of a single formulation or multiple formulations administered by the same or different routes.

In some embodiments, exemplary nonsteroidal anti-inflammatory drugs for co-administration with PRG4 or a fragment thereof include, but are not limited to, aspirin, celecoxib, diclofenac, diflunisal, etodolac, ibuprofen, indomethacin, ketorolac, ketoprofen, nabumetone, naproxen, oxaprozin, proxicam, salsalate, sulindac, tolmetin, and combinations thereof.

In some embodiments, PRG4 or a fragment thereof is co-administered with acetaminophen.

In some embodiments, exemplary opioids for co-administration with PRG4 or a fragment thereof include, but are not limited to, (dextro)propoxyphene, A-methylfentanyl, alfentanil, allylprodine, bezitramide, buprenorphine, butorphanol, carfentanyl, desmethylprodine, dextromoramide, dezocine, diacetylmorphine, dihydrocodeinone, dihydroetorphine, dimorphone, diphenoxylate, dipipanone, etorphine, fentanyl, ketobemidone, lefetamine, levacetylmethadol, levomethorphan, levorphanol, loperamide, meperidine, meptazinol, methadone, methylmorphine, morphine, nalbuphine, nalmefene, naloxone, naltrexone, nicomorphine, ohmefentanyl, oripavine, oxycodone, oxymorphone, PEPAP, paramorphine, pentazocine, phenazocine, piritramide, prodine, remifentanil, sufentanil, tapentadol, tilidine, tramadol, and combinations thereof.

In some embodiments, exemplary steroids for co-administration with PRG4 or a fragment thereof include, but are not limited to, prednisone and other corticosteroids.

In some embodiments, exemplary triptans include, but are not limited to, sumatriptan, zolmitriptan, naratriptan, rizatriptan, almotriptan, eletriptan, frovatriptan, and combinations thereof.

### 4. Diseases to be Treated

The disclosed compositions can be used to treat subjects suffering from acute and/or chronic pain. Such pain may arise from joint pain, pain associated with inflammatory bowel diseases, neuropathic pain, inflammatory pain, orofacial pain, migraine pain, cancer pain, ocular surface pain, Morton's neuroma pain, and other general pain diseases.

### Joint Pain

Pain originating from joints often originate in the area of the joint capsule or in the area of the joint close to the joint. Many etiologies may be considered, e.g., arthrotic or arthritic forms of disease, mechanical or other irritation of the joint surface near the bone, irritation or injury of the joint ligament structures, infections, autoimmune processes, etc. In all cases, which are of interest in the context of this disclosure, the resulting pain of nociceptive nerve fibers in the area close to the joints. Nociceptive nerve fibers are also referred to as C-fibers and A-delta fibers. If an analgesic substance (for example, local anesthetics or morphine) is injected into such a diseased joint, it alleviates the patient's discomfort. However, the substances used today have a limited duration of action, which is why the complaints usually return.

Accordingly, in some embodiments, the disclosed compositions and methods are used to treat a subject having joint pain. In certain embodiments, the joint pain is associated with arthritis, including osteoarthritis, rheumatoid arthritis (RA), juvenile idiopathic arthritis, fibromyalgia, gonococcal arthritis, gout, lupus, psoriatic arthritis, reactive arthritis, and/or septic arthritis.

In other embodiments the joint pain is associated with a temporomandibular joint (TMJ) disorder, such as TMJ arthritis, erosion of the joint, habitual grinding or clenching of the teeth, structural problems at birth, the use of orthodontic braces, poor posture that strains the muscles of the neck and face, prolonged stress, poor diet, and lack of sleep. In some embodiments, the joint pain is associated with dry mouth.

In yet other embodiments, the joint pain is associated with a strain, a sprain, a broken bone, a dislocation, or another injury.

In still other embodiments, the joint pain is associated with bursitis.

In other embodiments, the joint pain is associated with a meniscal tear, anterior cruciate ligament (ACL) tear, or a rotator cuff tear.

In still yet other embodiments, the joint pain also may be associated with Still's disease, ankylosing spondylitis, avascular necrosis, bone cancer, complex regional pain syndrome, hypothyroidism, leukemia, Lyme disease, osteomyelitis, Paget's disease of bone, polymyalgia rheumatica, pseudogout, rheumatic fever, rickets, sarcoidosis, and/or tendinitis.

### Inflammatory Bowel Disease Pain

Inflammatory bowel disease (IBD) is a term for two conditions (Crohn's disease and ulcerative colitis) that are characterized by chronic inflammation of the gastrointestinal (GI) tract. Prolonged inflammation results in damage to the GI tract. One of the symptoms of IBD is severe abdominal pain.

Accordingly, in some embodiments, the disclosed compositions and methods are used to treat a subject having pain associated with IBD. In some embodiments the subject has Crohn's disease. In other embodiments, the subject has ulcerative colitis.

### Bladder Pain Syndrome

Interstitial cystitis/bladder pain syndrome (IC/BPS) is a chronic painful bladder condition characterized by pelvic pain and urinary symptoms without another identifiable cause. BPS is a disorder that results in sensory dysregulation of bladder awareness, both in terms of pain perceived in the organ and an increased sense of urgency and/or frequency. It generally excludes the presence of other known causes of such symptoms: acute or recurrent infection, radiation- or medication-induced injury, malignancy, or nephrolithiasis.

Accordingly, in some embodiments, the disclosed compositions and methods are used to treat a subject having interstitial cystitis/bladder pain syndrome (IC/BPS).

### Neuropathic Pain

Neuropathic pain is caused by damage or injury to the nerves that transfer information between the brain and spinal cord from the skin, muscles, and other parts of the body.

The pain is usually described as a burning sensation and affected areas are often sensitive to the touch. Symptoms of neuropathic pain may also include excruciating pain, pins and needles, difficulty correctly sensing temperatures and numbness. Some patients find it hard to wear thick clothes as even slight pressure can aggravate the pain.

Neuropathic can occur as a result of alcoholism, amputation, chemotherapy, diabetes, facial nerve problems, HIV infection or AIDS, multiple myeloma, multiple sclerosis, nerve or spinal cord compression from herniated discs or from arthritis in the spine, shingles, spine surgery, syphilis, hand, foot and mouth disease, and/or thyroid problems.

Accordingly, in some embodiments, the disclosed compositions and methods are used to treat a subject having neuropathic pain. In some embodiments, the subject has neuropathic pain caused by nerve pressure or nerve damage after surgery or trauma, a viral infection, cancer, a vascular malformation, alcoholism, a neurological condition such as multiple sclerosis, and/or a metabolic condition such as diabetes. In some embodiments, the subject's neuropathic pain is a side effect of a medication.

### Inflammatory Pain

Acute inflammation can cause pain of varying types and severity. Pain may be constant and steady, throbbing and pulsating, stabbing, or pinching. Pain results when the buildup of fluid leads to swelling, and the swollen tissues push against sensitive nerve endings. Other biochemical processes also occur during inflammation. They affect how nerves behave, and this can contribute to pain.

Accordingly, in some embodiments, the disclosed compositions and methods are used to treat a subject having pain as a result of inflammation. In some embodiments, such inflammatory pain is acute inflammatory pain. In some embodiments, the inflammatory pain is constant.

### Orofacial Pain

Orofacial pain is a broad term used to describe symptoms of pain and/or dysfunction in the head and neck region. Multiple causes for orofacial pain may exist and the symptoms may include such diverse findings as headaches, neck pain, ear pain, dental pain, facial burning or stabbing sensations, and jaw joint pain. Symptoms may also include atypical pains or sensations such as ringing in the ears (tinnitus), dizziness, muscular incoordination or even abnormal itching or tingling in the head and neck region. The complaints may either develop gradually or have a rapid onset and can originate from neurovascular, neuropathic or musculoskeletal causes. Sources of orofacial pain include, but are not limited to, temporomandibular disorders (TMJDs), trigeminal neuropathic pain disorders, neurovascular disorders, complex temporomandibular disorders, burning mouth syndrome, sleep disorders, orofacial dyskinesias, mucositis, and dystonias, trauma, and cervicalgia.

Accordingly, in some embodiments, the disclosed compositions and methods are used to treat a subject having orofacial pain. In some embodiments, the orofacial pain is caused by a temporomandibular disorder (TMJD), a trigeminal neuropathic pain disorder, a neurovascular disorder, a complex temporomandibular disorder, burning mouth syndrome, a sleep disorder, orofacial dyskinesia and/or dystonia, trauma, and/or cervicalgia.

### Cancer Pain

In some aspects, cancer pain is caused by the cancer itself. Examples of cancer include, but are not limited to, anal cancer, bile duct cancer, bladder cancer, bone cancer, brain tumor and/or cancer, breast cancer, bronchial tumors, Burkitt lymphoma, cardiac tumors, cervical cancer, leukemia, colorectal cancer, uterine cancer, esophageal cancer, ewing sarcoma, fallopian tube cancer, gallbladder cancer, gastric cancer, gastrointestinal carcinoid tumor, head and neck cancer, kidney cancer, liver cancer, lip and oral cavity cancer, lung cancer, lymphoma, melanoma, skin cancer, metastatic cancer, mouth cancer, ovarian cancer, pancreatic cancer, prostate cancer, rectal cancer, salivary gland cancer, throat cancer, thyroid cancer or any combinations thereof. In one embodiment, the cancer pain happens if the cancer grows into or destroys nearby tissue. As a tumor grows, it can press on nerves, bones, or organs thereby causing pain. In another embodiment, the tumor can also release chemicals that can cause pain. In yet another embodiment, the cancer pain can be, but is not limited to, dull, achy, sharp, or burning. In yet another embodiment, the cancer pain can be, but is not limited to, constant, intermittent, mild, moderate, or severe.

Accordingly, in some embodiments, the disclosed compositions and methods are used to treat a subject having cancer pain.

### Migraine Pain

A migraine is a headache that can cause severe throbbing pain or a pulsing sensation, usually on one side of the head. It's often accompanied by nausea, vomiting, and extreme sensitivity to light and sound. Migraine attacks can last for hours to days, and the pain can be so severe that it interferes with a patient's daily activities.

Accordingly, in some embodiments, the disclosed compositions and methods are used to treat a subject having pain resulting from a migraine attack.

### Ocular Neuropathic Pain

Ocular neuropathic pain refers to a spectrum of disorders of ocular pain that are caused by damage or disease affecting the nerves. Ocular neuropathic pain is frequently associated with damaged or dysfunctional corneal nerves, but the condition can also be caused by peripheral or centralized sensitization. The condition shares some characteristics with somatic neuropathic pain in that it is similarly associated with abnormal sensations (dysesthesia) or pain from normally non-painful stimuli (allodynia).

This condition is frequently associated with dry eye disease since sensations of dryness and burning in the eye are a common symptom of both neuropathic eye pain and dry eye, but ocular neuropathic pain should be considered as a disease in its own right. Neuropathic pain patients may have little or no signs of aqueous dry eye, and frequently respond poorly to conventional dry eye treatments. Unlike conventional dry eye disease, there may be little or no sign of ocular surface damage, (the condition is sometimes referred to as “pain without stain”); however, patients may also have symptoms of dry eye but with pain symptoms that are out of proportion to the dry eye presentation.

The experience of painful sensations in this condition can vary widely, reflecting a variety of causal factors such as: types of noxious stimuli causing insult to ocular surface nociceptors, the types of corneal sensory receptors affected—including cold-sensing thermoreceptors, mechanoreceptors, and polymodal receptors—the extent of the inflammatory responses, and the type or types of disorders and damage affecting the nervous system.

Accordingly, in some embodiments, the disclosed compositions and methods are used to treat a subject having ocular neuropathic pain. In some embodiments, the ocular neuropathic pain is associated with damaged or dysfunctional corneal nerves. In other embodiments, the ocular neuropathic pain is caused by peripheral or centralized sensitization. In some embodiments, the subject having ocular neuropathic pain also has dry eye.

### Burn Pain

All burn injuries are painful. First-degree or very superficial partial-thickness burns may damage only the outer layers of the skin (the epidermis) but they cause mild pain and discomfort, especially when something such as clothing rubs against the burned area. Moderate to deep partial-thickness or second-degree burns result in variable amounts of pain depending on the amount of destruction of the dermis. Superficial dermal burns are initially the most painful. Even the slightest change in the air currents moving past the exposed superficial dermis causes a patient to experience excruciating pain. Without the protective covering of the epidermis, nerve endings are sensitized and exposed to stimulation. In addition, as the inflammatory response progresses, with the increase in swelling and the release of vasoactive substances, pain is bound to increase.

Accordingly, in some embodiments, the disclosed compositions and methods are used to treat a subject having pain associated with a burn. In some embodiments, the burn is a first-degree or superficial partial-thickness burn. In some embodiments, the burn is a second-degree or moderate to deep partial thickness burn. In some embodiments, the burn is a superficial dermal burn.

### Morton's Neuroma Pain

Morton's neuroma is a benign (non-cancerous) tumor of one or more nerves. Specifically, Morton's neuroma is not actually a tumor, but a thickening of the tissue that surrounds the digital nerve leading to the toes. This thickening can lead to various levels of pain or discomfort in the forefoot that may limit the ability to perform certain activities. Morton's neuroma can produce symptoms of sharp burning pain in the ball of foot, stinging pain, tingling or numbness in the affected toes, a feeling like standing on pebbles. In one embodiment, the ball of foot which is affected by Morton's neuroma is commonly the area between the third and fourth toes.

Accordingly, in some embodiments, the disclosed compositions and methods are used to treat a subject having Morton's neuroma pain. In some embodiments, the Morton's neuroma pain is associated with a thickening of the tissue that surrounds the digital nerve leading to the toes.

### General Pain Diseases

Most pain subsides after an injury heals or an illness runs its course, but with chronic pain syndrome, pain can last for months and even years after the body heals. It can even occur when there's no known trigger for the pain. Chronic pain is defined as lasting anywhere from 3- to 6-months, and it affects some 25 million Americans. Chronic pain syndrome takes a toll on both your physical and mental health. While the pain can be near-constant, there may be flares of more intense pain due to increases in stress or activity. Symptoms include joint pain, muscle aches, burning pain, fatigue, sleep problems, loss of stamina and flexibility due to decreased activity, mood problems (including depression, anxiety, and irritability). Conditions that cause widespread and long-lasting pain. Some of these conditions include, but are not limited to, chronic pelvic pain, endometriosis pain, osteoarthritis, rheumatoid arthritis (RA), back pain, fibromyalgia, inflammatory bowel disease (IBD), surgical trauma, and advanced cancer.

Accordingly, in some embodiments, the disclosed compositions and methods are used to treat a subject having a general pain disease. In some embodiments, the general pain disease is a chronic pain syndrome. In some embodiments, the chronic pain syndrome is associated with osteoarthritis, rheumatoid arthritis (RA), back pain, fibromyalgia, inflammatory bowel disease (IBD), surgical trauma, and/or advanced cancer. In some embodiments, the chronic pain is chronic pelvic pain. In some embodiments, the chronic pain syndrome is associated with endometriosis.

### 5. Administration of PRG4

While PRG4 is produced naturally within the body, the effects of the disclosure are observed when exogenous PRG4 or a fragment thereof is administered to a subject. Accordingly, in one embodiment, the PRG4 or fragment thereof administered to a subject is exogenous human PRG4. In other embodiments, the PRG4 or fragment thereof administered to the patient is recombinant human PRG4 (rhPRG4).

In some embodiments, the pharmaceutically effective amount of PRG4 or a fragment thereof administered will depend on variables such as the severity of the pain subject is experiencing. For example, the amount of PRG4 or fragment thereof administered in a subject may be based on the level of joint pain from arthritis (e.g., rheumatoid arthritis), pain associated with inflammatory bowel diseases (e.g., ulcerative colitis, Crohn's disease) neuropathic pain, inflammatory pain, orofacial pain, cancer pain, pain associate with a migraine, ocular surface pain experienced by the subject. In some embodiments, additional factors that are considered in determining the amount of PRG4 or fragment thereof to administer to a given subject are the overall health of the patient, the pharmaceutical formulation, and the route of administration. In one embodiment, the initial dosage can be increased beyond the upper level in order to rapidly achieve the desired blood-level or tissue level. Alternatively, in another embodiment, the initial dosage can be smaller than the optimum, and the dosage may be progressively increased during the course of treatment. In still another embodiment, the optimal dose can be determined by routine experimentation.

In some embodiments, the pharmaceutically effective amount of PRG4 or fragment thereof is administered in an amount that is insufficient to provide boundary lubrication, but sufficient to treat joint pain or allodynia. In one embodiment, the PRG4 or fragment thereof is administered in an amount that is insufficient to provide boundary lubrication, but sufficient to reduce inflammation associated with sickle cell disease. Accordingly, in some embodiments, a pharmaceutically or therapeutically effective amount of PRG4 or fragment thereof for administration according to the disclosure is in the range of about 0.1 μg/kg to about 4000 μg/kg, or about 0.1 μg/kg to about 1000 μg/kg, or about 0.1 μg/kg to about 100 μg/kg, or about 0.1 μg/kg to about 50 μg/kg. In some embodiments, the therapeutically effective amount of PRG4 or fragment thereof administered is in the range of about 0.1 mg/kg to about 100 mg/kg, or about 1 mg/kg to about 100 mg/kg, or about 1 mg/kg to about 10 mg/kg. The PRG4 or fragment thereof administered may also be in a range of about 0.1 μg/mL to about 30 mg/mL, or about 1 μg/mL to about 10 mg/mL, or about 10 μg/mL to about 1 mg/mL. In some embodiments, PRG4 or fragment thereof is administered at concentrations no greater than about 60 μg/mL. In some embodiments, PRG4 or fragment thereof is administered in small volumes of about 1 μL to 100 μL per dose.

In further embodiments, lubricin or fragment thereof is administered locally or systemically in an amount sufficient to achieve a concentration of lubricin or fragment thereof in a synovial fluid of a joint of a subject of at least about 50 μg/mL, at least about 100 μg/mL, at least about 150 μg/mL, at least about 200 μg/mL, at least about 250 μg/mL, at least about 300 μg/mL, at least about 350 μg/mL, at least about 400 μg/mL, at least about 450 μg/mL, at least about 500 μg/mL, at least about 550 μg/mL, at least about 600 μg/mL, at least about 650 μg/mL, at least about 750 μg/mL, at least about 800 μg/mL, at least about 850 μg/mL, at least about 900 μg/mL, at least about 950 μg/mL, or at least about 1000 μg/mL. It is contemplated that to achieve a concentration of lubricin or fragment thereof in the synovial fluid of a joint of a subject, lubricin or fragment thereof must be administered to the subject at a concentration higher than the desired concentration of lubricin in the synovial fluid. In certain embodiments, a total amount of 2 mg to 10 mg of lubricin or fragment thereof is administered per dose, e.g., about 2 mg to about 10 mg, about 2 mg to about 5 mg, about 2 mg to about 3 mg, about 3 mg to about 4 mg, about 4 mg to about 5 mg, about 5 mg to about 6 mg, about 6 mg to about 7 mg, about 7 mg to about 8 mg, about 8 mg to about 9 mg, about 9 mg to about 10 mg, or about 5 mg to about 10 mg. In certain embodiments, more than about 10 mg of lubricin is administered per dose. In some embodiments, the lubricin is administered intra-articularly to the joint to achieve the desired concentration of PRG4 in the synovial fluid. It is contemplated in this disclosure that the dose of PRG4 used for intravenous administration is at least about 1.5-fold, or at least about 2-fold, or at least about 3-fold, or at least about 4-fold, or at least about 5-fold, or at least about 10-fold higher than dose used for intra-articular administration. For example, in one embodiment, PRG4 is administered to a patient suffering from gout wherein PRG4 is administered in the amount about 0.05 mg/kg to about 1.50 mg/kg.

In some embodiments, the current disclosure contemplates that PRG4 or fragment thereof can be administered to the patient in pain, wherein the administration is either systemic or local administration. In one embodiment, local administration, for example, includes intra-articular administration into an affected joint or injection into an affected area in which a subject is experiencing pain. For example, in some embodiments, injection may be directly into an affected joint such as a hip, shoulder, elbow, knee, toe, finger, ankle, or wrist. In some other embodiments, injection into an affected area such as the instep or heel of the foot is contemplated. In another embodiment, alternatively, local administration also can also include, but is not limited to, intraocular, intrathecal, intravesical, intravitreal, and perivascular administration into an affected area in which a subject is experiencing pain. Accordingly, in one embodiment, a pharmaceutically or therapeutically effective amount of PRG4 or fragment thereof for local administration according to the disclosure may be in the range of about 0.1 μg/kg to about 4000 μg/kg, or about 0.1 μg/kg to about 1000 μg/kg, or about 0.1 μg/kg to about 100 μg/kg, or about 0.1 to about 50 μg/kg. PRG4 administered may also be in a range of about 0.1 μg/mL to about 30 mg/mL, or about 1 μg/mL to about 10 mg/mL, or about 10 μg/mL to about 1 mg/mL. In another embodiment, the PRG4 or fragment thereof administered may also be in an amount of about 2 mg to about 10 mg, about 2 mg to about 5 mg, about 5 mg to about 10 mg or greater than about 10 mg. In still another embodiment, these administrations may be carried out every day, every other day, every three days, every four days, every five days, every six days, once weekly, once every other week, once every third week, or once monthly per treatment cycle.

Systemic administration of PRG4 or fragment thereof is also contemplated by some embodiments of the disclosure. For example, PRG4 or a fragment thereof can be systemically administered in an enteral manner, such as oral, rectal, sublingual, sublabial, or buccal delivery. PRG4 or a fragment thereof may be systemically administered in a parenteral manner, such as nasal, by inhalation, intraperitoneal, intravenous, intramuscular, subcutaneous, intradermal, transdermal, or transmucosal delivery.

In some embodiments, one route of systemic administration of PRG4 or a fragment thereof contemplated herein is intravenous administration. The optimal dose can be determined by routine experimentation depending on variables such as the level of pain the patient is experiencing, the overall health of the patient, and the pharmaceutical formulation. In some embodiments, for systemic administration, a dose between about 0.1 mg/kg and about 100 mg/kg, alternatively between about 0.5 mg/kg and about 50 mg/kg, alternatively, between about 1 mg/kg and about 25 mg/kg, alternatively between about 2 mg/kg and about 10 mg/kg, alternatively between about 5 mg/kg and about 10 mg/kg, alternatively between about 0.05 mg/kg and about 1.50 mg/kg is administered and may be given, for example, once daily, once weekly, twice weekly, three times weekly, once every other week, once every third week, or once monthly per treatment cycle.

In some embodiments, for therapeutic use, the PRG4 or fragment thereof administered is combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. In some embodiments, pharmaceutically acceptable carriers include buffers, solvents, dispersion media, coatings, isotonic and absorption delaying agents, and the like, that are compatible with pharmaceutical administration. In one embodiment, suitable carriers include phosphate buffered saline at concentrations ranging from about 1 μg/mL to about 1000 μg/mL, and in some aspects about 100 μg/mL to about 500 μg/mL. In another embodiment, suitable carriers may also include physiological saline, bacteriostatic water, Cremophor EL™ (BASF, Parsippany, N.J.) or phosphate buffered saline (PBS), optionally in admixture with surfactants such as polysorbates. In yet another embodiment, suitable carriers may also include a solvent or dispersion medium containing, for example, water, ethanol, polyol (for example, glycerol, propylene glycol, and liquid polyethylene glycol), and suitable mixtures thereof. The carrier should be stable under the conditions of manufacture and storage and should be preserved against microorganisms. The use of carriers for pharmaceutically active substances is known in the art. For example, see Remington's Pharmaceutical Sciences, 18th ed. (Mack Publishing Company, 1990).

In some embodiments, useful formulations can be prepared by methods well known in the pharmaceutical arts. For example, see Remington's Pharmaceutical Sciences, 18^{th} ed. (Mack Publishing Company, 1990). Formulation components suitable for parental administration include a sterile diluent such as water for injection, saline solution, fixed oils, polyethylene glycol, glycerin, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose. In some embodiments, Lubricin for administration can be present in a dosage unit form and can be prepared by any suitable method and should be formulated to be compatible with its intended route of administration.

In some embodiments, PRG4 or a fragment thereof for administration should be formulated to be compatible with its intended route of administration, for example, intra-articular (IA), intravenous (IV), intramuscular, subcutaneous, intradermal, intranasal, transdermal, topical, transmucosal, oral, and rectal administration. In some embodiments, the formulation of PRG4 or a fragment thereof can be presented in a dosage unit form and prepared by any suitable method known in the art. For example, formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose. In some aspects, the dosage unit form can be for immediate release delivery, sustained release delivery, controlled release delivery, or any combinations thereof.

In some embodiments, pharmaceutical formulations for PRG4 or a fragment thereof are sterile. Sterilization can be accomplished, for example, by filtration through sterile filtration membranes. Where the composition is lyophilized, filter sterilization can be conducted prior to or following lyophilization and reconstitution. Aqueous solutions may be packaged for use as-is, or lyophilized, the lyophilized preparation being combined with a sterile aqueous carrier prior to administration. The pH of the preparations typically is between 3 and 11, more preferably between 5 and 9 or between 6 and 8, and most preferably between 7 and 8, such as 7 to 7.5. Formulated PRG4 or a fragment thereof for administration may be packaged in multiple single dose units, each containing a fixed amount of the above-mentioned agent or agents, such as in a sealed package of tablets or capsules.

In some embodiments, pharmaceutical compositions containing PRG4 or a fragment thereof, such as those disclosed herein, can be presented in a dosage unit form and can be prepared by any suitable method. In some embodiments, a pharmaceutical composition should be formulated to be compatible with its intended route of administration. Examples of routes of administration are intravenous (IV), intradermal, inhalation, transdermal, topical, transmucosal, and rectal administration. In some embodiments, the pharmaceutical compositions are intended for parenteral, intranasal, topical, oral, or local administration, such as by a transdermal means, for therapeutic treatment. The pharmaceutical compositions can be administered parenterally (e.g., by intravenous, intramuscular, or subcutaneous injection), or by oral ingestion, or by topical application or intra-articular injection at areas affected by gout such as the knee, ankle, finger joint, or elbow. Additional routes of administration include intravascular, intra-arterial, intratumor, intraperitoneal, intraventricular, intraepidural, as well as nasal, ophthalmic, intrascleral, intraorbital, rectal, topical, or aerosol inhalation administration.

In one embodiment, the disclosure provides compositions for parenteral administration that comprise the above-mentioned agents dissolved or suspended in an acceptable carrier, such as an aqueous carrier, e.g., water, buffered water, saline, PBS, and the like. In another embodiment, the compositions may contain pharmaceutically acceptable auxiliary substances as required to approximate physiological conditions, such as pH adjusting and buffering agents, tonicity adjusting agents, wetting agents, detergents, and the like. In yet another embodiment, the disclosure also provides compositions for oral delivery, which may contain inert ingredients such as binders or fillers for the formulation of a tablet, a capsule, and the like. In still another embodiment, this disclosure provides compositions for local administration, which may contain inert ingredients such as solvents or emulsifiers for the formulation of a cream, an ointment, and the like.

In one embodiment, a route of administration for PRG4 is by IV infusion. Useful formulations can be prepared by methods well known in the pharmaceutical art. For example, see Remington's Pharmaceutical Sciences, 18^{th} ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl paraben; antioxidants such as ascorbic acid or sodium bisulfite; chelating agents such as EDTA; buffers such as acetates, citrates or phosphates; and agents for the adjustment of tonicity such as sodium chloride or dextrose.

All references and publications included herein are incorporated by reference. The following examples are not intended to be limiting.

### EXEMPLIFICATION

### Example 1: rhPRG4 Binds TrkA and Decreases TNF Induced Expression of NGF in Corneal Epithelial Cells

### 1. Surface Plasmon Resonance

### Materials

rhPRG4
TrkA (sample-#175-TK, Lot-ERI0617071, R&D)

### Equipment

Biacore X100 instrument
CM5 sensor chips

### Methods

50 μg TrkA sample was reconstituted in PBS to prepare 100 μg/mL stock solution according to manufacturer's instruction then diluted (1:1) in 20 mM sodium acetate solution (pH 5 and 5.5) to prepare immobilization solution (50 μg/mL in 10 mM sodium acetate).

### 2. SPR Results

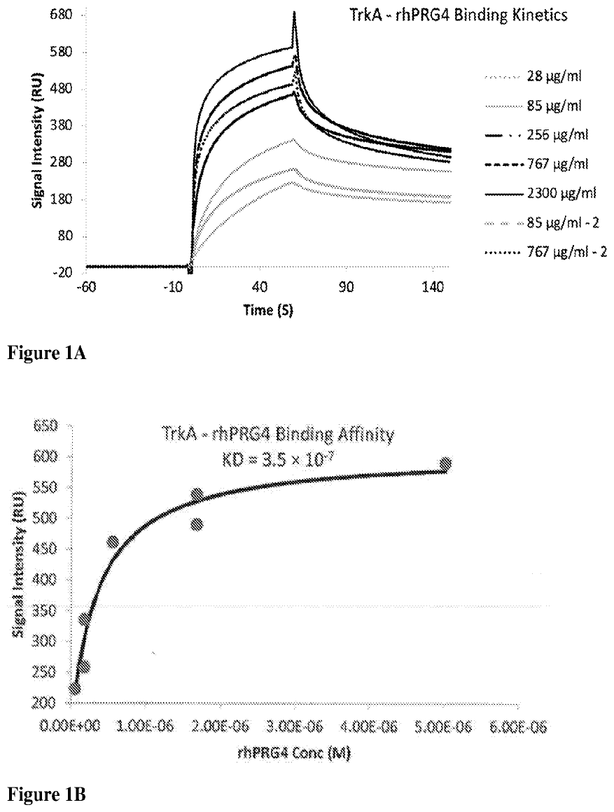

A new CM5 chip was coated with TrkA using 50 μg/mL solution in 10 mM sodium acetate (tried two different pH solutions 5 and 5.5). After several attempts, only about 50 RU signal was immobilized. Although the immobilization was difficult, binding of rhPRG4 looked very good (FIGS. 1A and 1B). Indeed, rhPRG4 binds TrkA about10⁻⁷ M affinity (FIG. 1B). After determining binding and regeneration buffer kinetics experiment was set using 1M NaCl 200 S injection for regeneration.

Table 1 shows RNASeq on human corneal epithelial cells: +/−10 ng/mL TNF, +/−300 μg/mL rhPRG4.

TABLE 1

| Ensembl- | Hgnc- | Log2FoldChnage- | Padj_TNFa\| | Log2FoldChnage- | Padj_Lub\| | Log2FoldChnage- | Padj_Lub\| |
|---|---|---|---|---|---|---|---|
| gene-id3 | symbol | TNF\|NoLub | NoLub | Lub\|TNFa | TNFa | Lub\|NoTNFa | NoTNFa |
| ENSG00000134259 | NGF | 1.69 | 0.02 | −1.74 | 0.04 | −1.14 | 0.40 |
| ENSG00000104321 | TRPA | 2.49 | 1.44E−13 | −0.87 | 0.02 | 0.30 | 0.69 |
| | 1 | | | | | | |

### Example 2: NGF and TrkA Expression in TMJ of PRG4 Deficient (GT) Mice: Effect of Re-Expression by Tamoxifen (TMX)

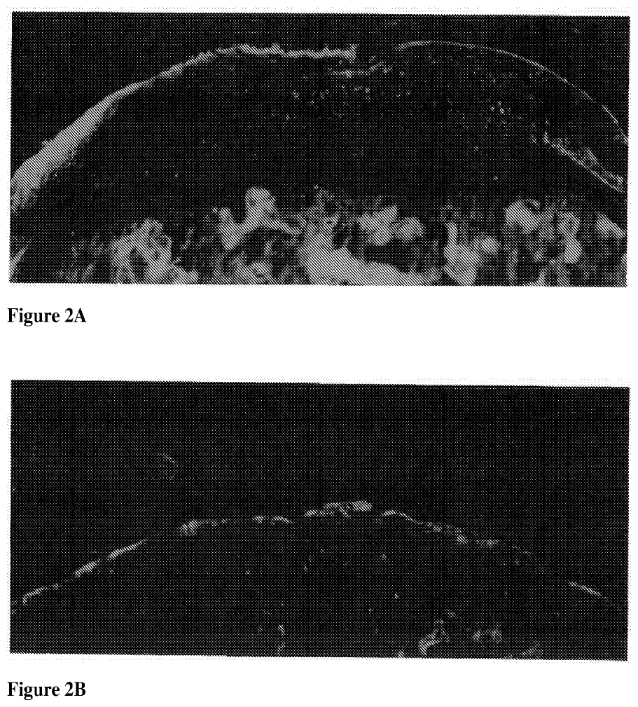

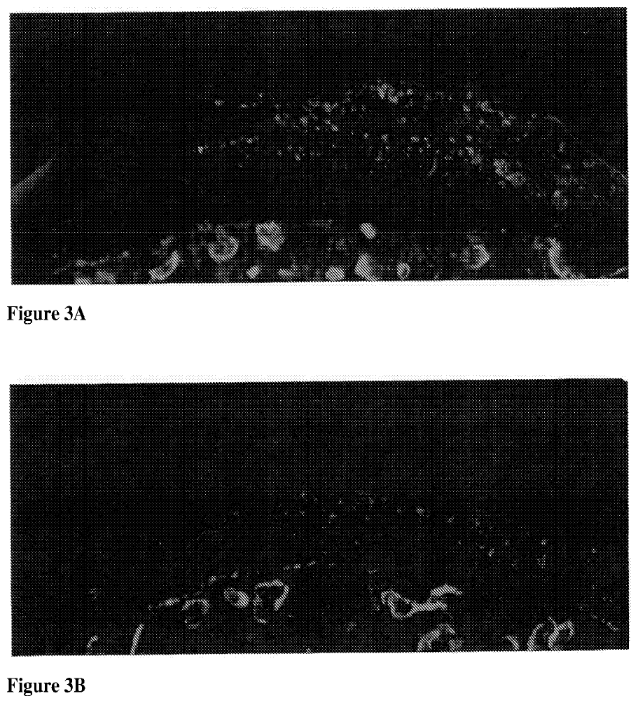

PRG4 deficient Mice (GT) at 6 months were compared to TMX treated mice (GTT) at 3 weeks to re-express PRG4. PRG4 deficient Mice (GT) increased expression and localization of NGF (FIGS. 2A and 2B) and increased expression of TrkA (FIGS. 3A and 3B) as compared to the TMX treated mice (GTT).

### Example 3: Fura2 Calcium Imaging Study to Assess the Effect of Lubricin on Sensory Ganglion Neurons

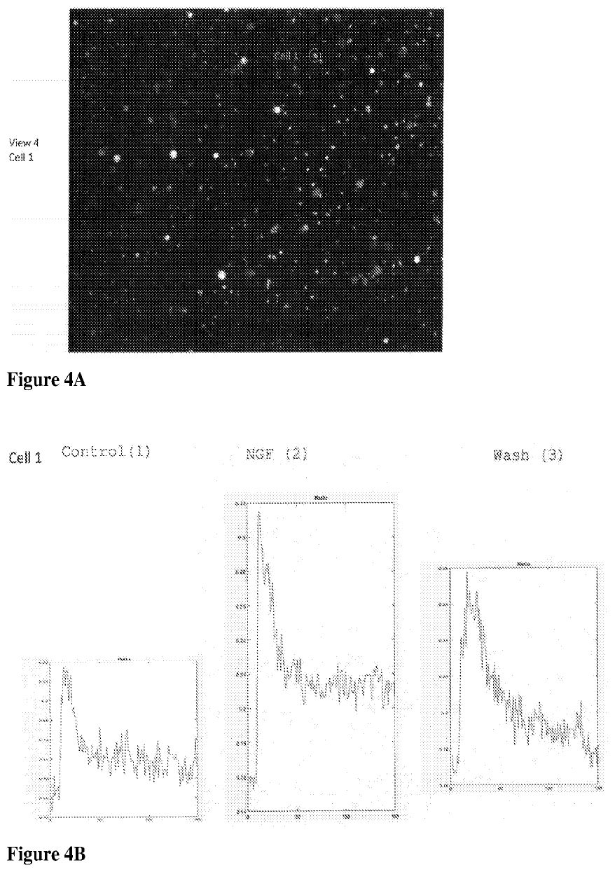

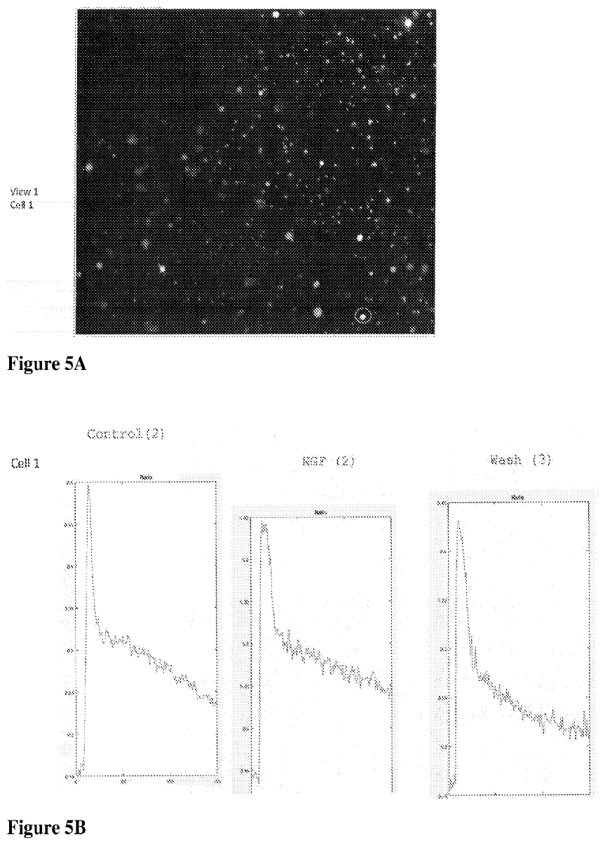

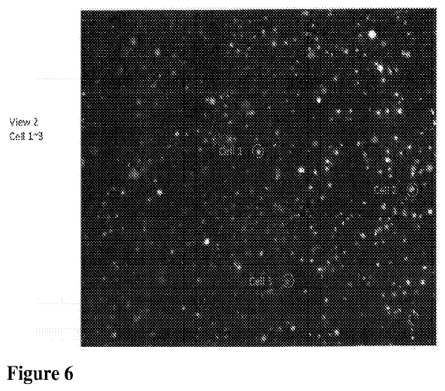

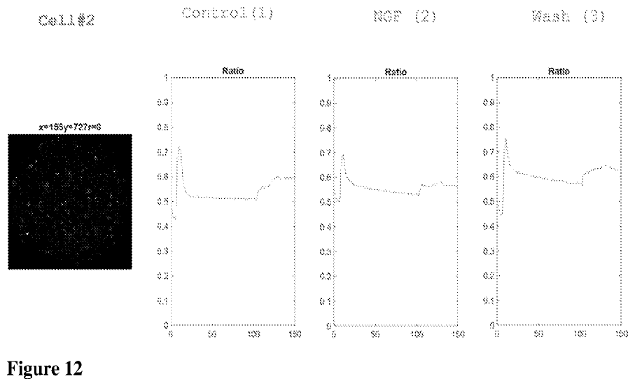

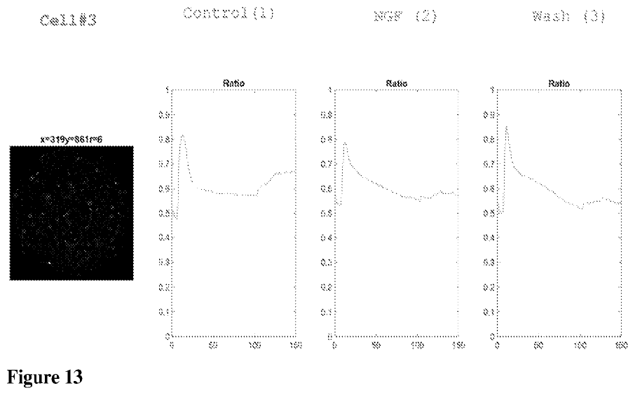

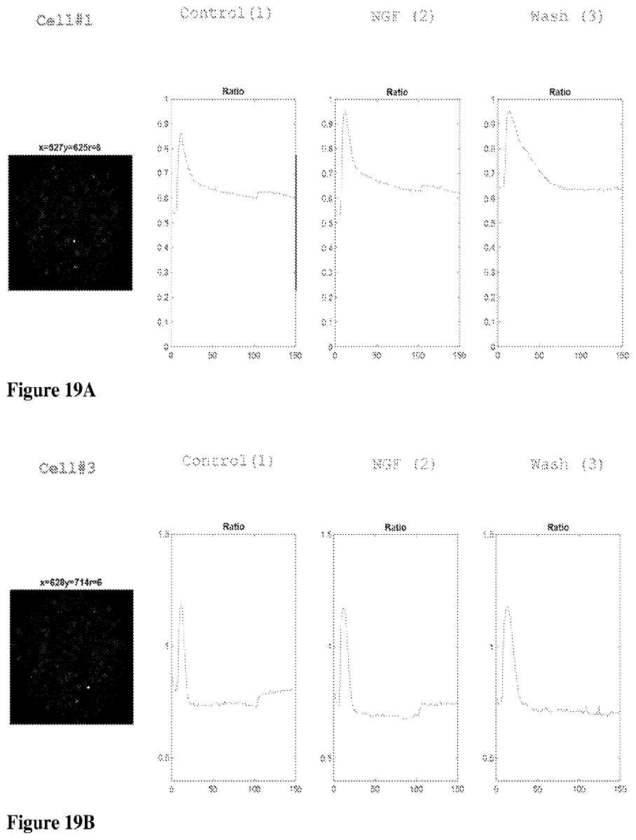

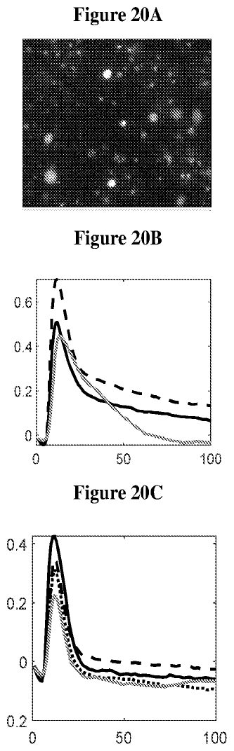

Capsaicin (CAP) response is sensitized by NGF application. A robust NGF sensitization of CAP response (increased peak response) was observed (FIGS. 4B, 19A and 20B). NGF sensitization of CAP response (increased decay time constant) was also observed (FIGS. 5B, 12, 13 and 19B).

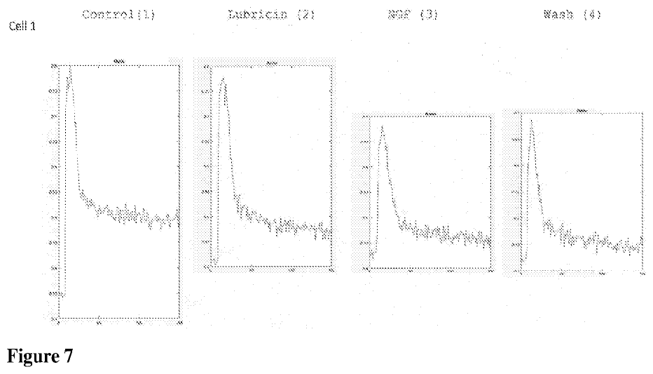

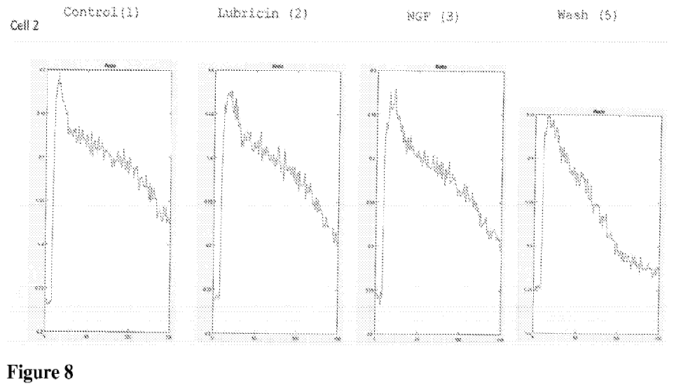

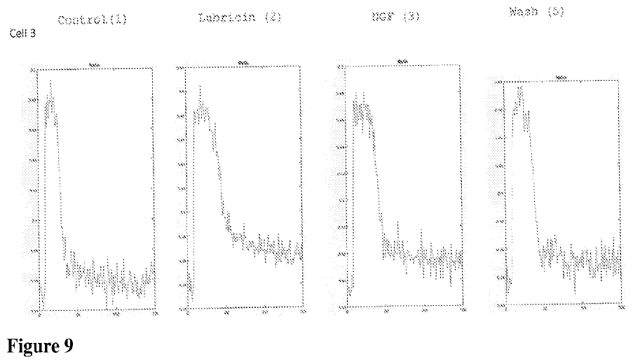

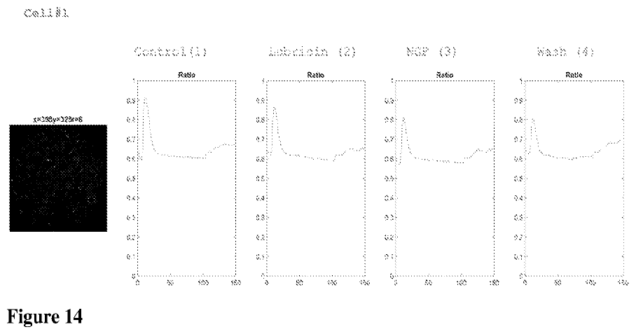

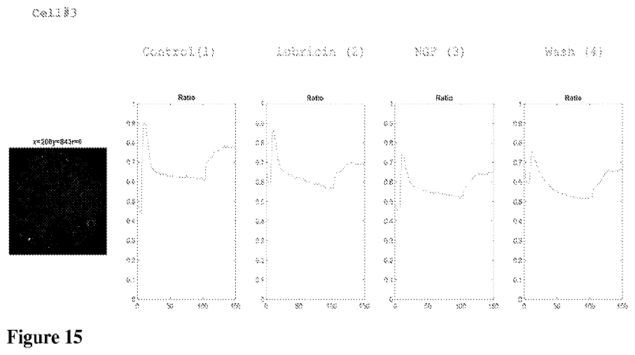

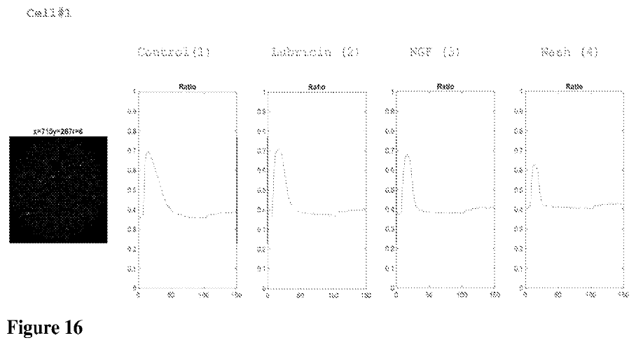

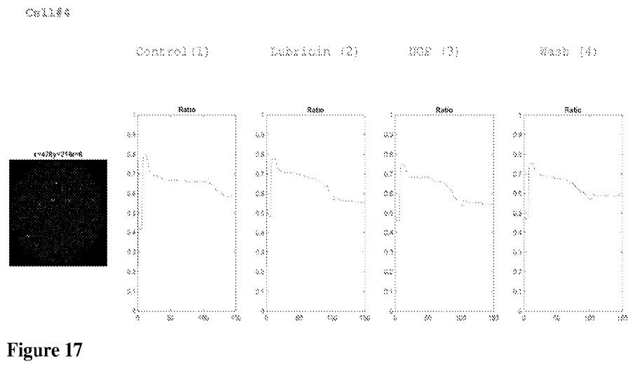

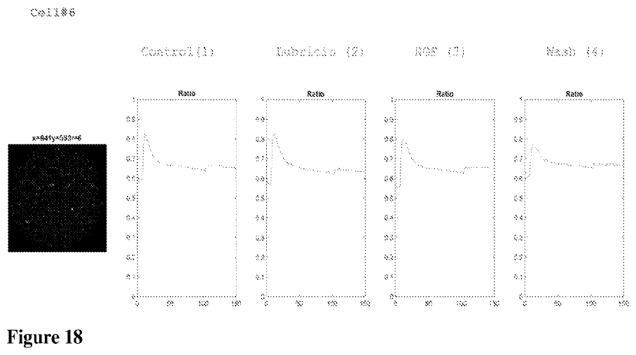

Also observed was that NGF sensitization of CAP response was blunted by the pre-application of Lubricin (FIGS. 7-9, 14-18, 20C). Thus, FIGS. 7-9, 14-18, and 20C show that the pre-treatment with lubricin prevents the sensitization effect of NGF.

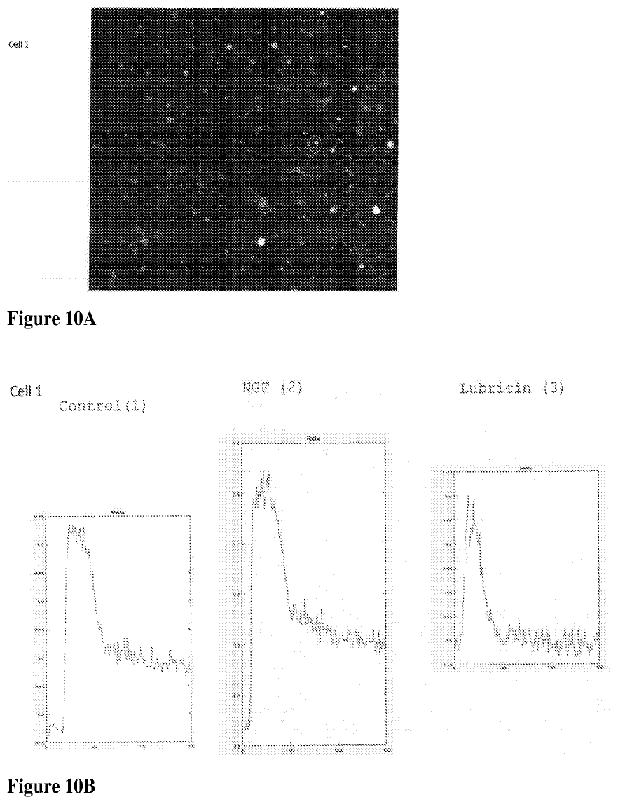

NGF sensitization of CAP response also was slighted reduced by post-application of Lubricin (FIG. 10B).

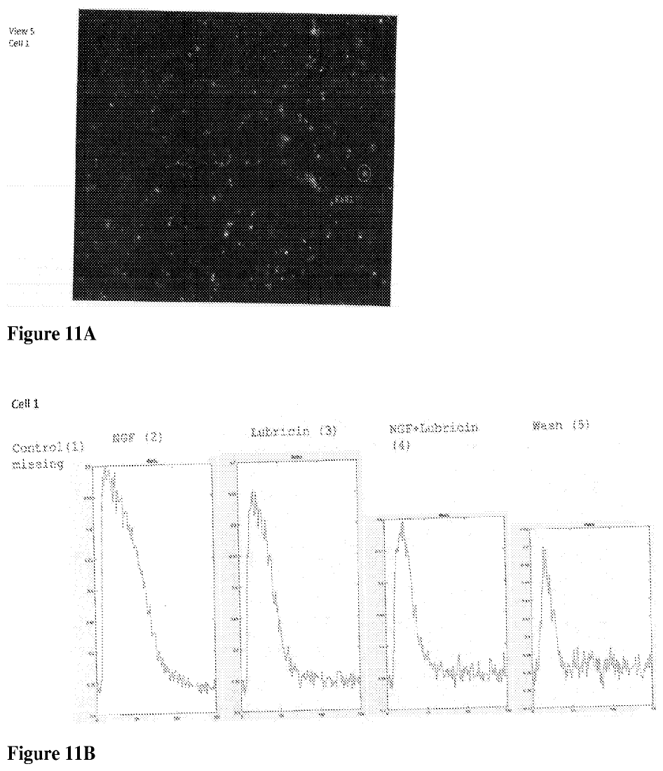

NGF sensitization of CAP response was prevented when NGF and lubricin were added together (FIG. 11B).

### Example 4: Von Frey Test to Measure Pain Sensitivity

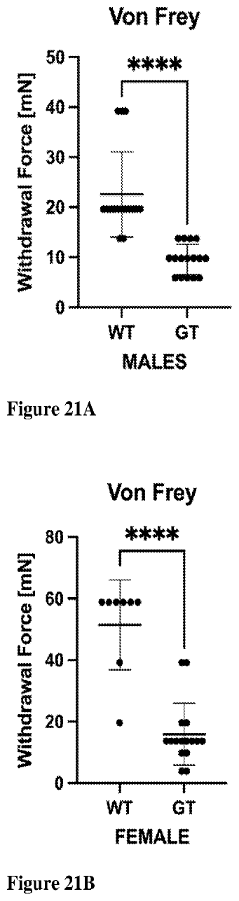

The Von Frey Test was performed to prove the effect of PRG4 on pain sensitivity. In one embodiment, the Von Frey Test involves applying a punctate stimulus to given region of the subject's body and recording the stimulus intensity that evokes a withdrawal reflex. In this experiment, PRG4 deficient mice (GT) was compared to wild type mice (WT). The results from the pain measurement data on both male (FIG. 21A) and female (FIG. 21B) show that PRG4 deficient mice (GT) have increased pain sensitivity compared to wild type mice (WT).

1. A method of treating pain, wherein the method comprises administering a pharmaceutically effective amount of PRG4 to a subject in need thereof.

2. The method of claim 1, wherein the PRG4 is rhPRG4.

3. The method of claim 1, wherein the PRG4 is administered to the subject as a pharmaceutically acceptable composition.

4. The method of claim 3, wherein the pharmaceutically acceptable composition comprises a pharmaceutically acceptable carrier or diluent.

5. The method of claim 1, wherein the pain is joint pain.

6. The method of claim 5, wherein the joint pain is associated with arthritis, including osteoarthritis, rheumatoid arthritis (RA), juvenile idiopathic arthritis, fibromyalgia, gonococcal arthritis, gout, lupus, psoriatic arthritis, reactive arthritis, septic arthritis, or a combination thereof.

7. The method of claim 5, wherein the joint pain is associated with a temporomandibular joint (TMJ) disorder or dry mouth.

8. The method of claim 7, wherein the temporomandibular joint (TMJ) disorder is TMJ arthritis, erosion of the joint, habitual grinding or clenching of the teeth, structural problems at birth, the use of orthodontic braces, poor posture that strains the muscles of the neck and face, prolonged stress, poor diet, lack of sleep, or a combination thereof.

9. The method of claim 5, wherein the joint pain is associated with a strain, a sprain, a broken bone, or a dislocation.

10. The method of claim 5, wherein the joint pain is associated with bursitis.

11. The method of claim 5, wherein the joint pain is associated with a meniscal tear, anterior cruciate ligament (ACL) tear, or a rotator cuff tear.

12. The method of claim 5, wherein the joint pain is associated with Still's disease, ankylosing spondylitis, avascular necrosis, bone cancer, complex regional pain syndrome, hypothyroidism, leukemia, Lyme disease, osteomyelitis, Paget's disease of bone, polymyalgia rheumatica, pseudogout, rheumatic fever, rickets, sarcoidosis, tendinitis, or a combination thereof.

13. The method of claim 1, wherein the pain is inflammatory bowel disease pain.

14. The method of claim 13, wherein the subject has Crohn's disease.

15. The method of claim 13, wherein the subject has ulcerative colitis.

16. The method of claim 1, wherein the subject has interstitial cystitis/bladder pain syndrome (IC/BPS).

17. The method of claim 1, wherein the pain is neuropathic pain.

18. The method of claim 17, wherein the subject has neuropathic pain caused by nerve pressure or nerve damage after surgery or trauma, a viral infection, cancer, a vascular malformation, alcoholism, a neurological condition such as multiple sclerosis, a metabolic condition, hand, foot and mouth disease, or a combination thereof.

19. The method of claim 18, wherein the subject has neuropathic pain caused by metabolic condition, wherein the metabolic condition is diabetes.

20. The method of claim 17, wherein the neuropathic pain is a side effect of a medication.

21. The method of claim 1, wherein the pain is inflammatory pain.

22. The method of claim 21, wherein the inflammatory pain is acute inflammatory pain.

23. The method of claim 21, wherein the inflammatory pain is constant.

24. The method of claim 1, wherein the pain is orofacial pain.

25. The method of claim 24, wherein the orofacial pain is caused by a temporomandibular disorder (TMJD), a trigeminal neuropathic pain disorder, a neurovascular disorder, a complex temporomandibular disorder, burning mouth syndrome, a sleep disorder, orofacial dyskinesia and/or dystonia, trauma, cervicalgia, mucositis, or a combination thereof.

26. The method of claim 1, wherein the pain is migraine pain.

27. The method of claim 1, wherein the pain is cancer pain.

28. The method of claim 1, wherein the pain is ocular neuropathic pain.

29. The method of claim 28, wherein the ocular neuropathic pain is associated with damaged or dysfunctional corneal nerves.

30. The method of claim 28, the ocular neuropathic pain is caused by peripheral or centralized sensitization.

31. The method of claim 28, wherein the subject having ocular neuropathic pain also has dry eye.

32. The method of claim 1, wherein the pain is Morton's neuroma pain.

33. The method of claim 32, wherein the Morton's neuroma pain is associated with a thickening of the tissue around one of the nerves leading to toes.

34. The method of claim 1, wherein the pain is a general pain disease.

35. The method of claim 34, wherein the general pain disease is a chronic pain syndrome.

36. The method of claim 35, wherein the chronic pain syndrome is associated with osteoarthritis, rheumatoid arthritis (RA), back pain, fibromyalgia, inflammatory bowel disease (IBD), surgical trauma, advanced cancer, or a combination thereof.

37. The method of claim 35, wherein the chronic pain syndrome is chronic pelvic pain.

38. The method of claim 35, wherein the chronic pain syndrome is associated with endometriosis.

39. A method of inhibiting tumor necrosis factor (TNF)-α simulated nerve growth factor (NGF) by administering a pharmaceutically effective amount PRG4 to a subject in need thereof.

40. The method of claim 39, wherein the PRG4 is rh PRG4.

41. The method of claim 39, wherein the PRG4 is administered to the subject as a pharmaceutically acceptable composition.

42. The method of claim 41, wherein the pharmaceutically acceptable composition comprises a pharmaceutically acceptable carrier or diluent.

43. A method of inhibiting transient receptor potential ankyrin 1 (TrpA1) expression by administering a pharmaceutically effective amount PRG4 to a subject in need thereof.

44. The method of claim 43, wherein the PRG4 is rhPRG4.

45. The method of claim 43, wherein the PRG4 is administered to the subject as a pharmaceutically acceptable composition.

46. The method of claim 45, wherein the pharmaceutically acceptable composition comprises a pharmaceutically acceptable carrier or diluent.

47. A method of inhibiting nerve growth factor (NGF)/tropomyosin receptor kinase A (Trk A) binding by administering PRG4.

48. The method of claim 47, wherein the PRG4 is rhPRG4.

49. The method of claim 47, wherein the PRG4 is administered to the subject as a pharmaceutically acceptable composition.

50. The method of claim 49, wherein the pharmaceutically acceptable composition comprises a pharmaceutically acceptable carrier or diluent.
